SEQUENCE LISTING (1 sequence)

SEQ ID NO: 1 - protein, 457 aa, Homo sapiens
MTTDEGAKNNEESPTATVAEQGEDITSKKDRGVLKIVKRVGNGEETPMIGDKVYVHYKGKLSNGKKFDSSHDRNEPFVFSLGKGQVIKAWDIGVATMKKGEICHLLCKPEYAYGSAGSLPKIPSNATLFFEIELLDFKGEDLFEDGGIIRRTKRKGEGYSNPNEGATVEIHLEGRCGGRMFDCRDVAFTVGEGEDHDIPIGIDKALEKMQREEQCILYLGPRYGFGEAGKPKFGIEPNAELIYEVTLKSFEKAKESWEMDTKEKLEQAAIVKEKGTVYFKGGKYMQAVIQYGKIVSWLEMEYGLSEKESKASESFLLAAFLNLAMCYLKLREYTKAVECCDKALGLDSANEKGLYRRGEAQLLMNEFESAKGDFEKVLEVNPQNKAARLQISMCQKKAKEHNERDRRIYANMFKKFAEQDAKEEANKAMGKKTSEGVTNEKGTDSQAMEEEKPEGHV

(19) United States
(12) Patent Application Publication
(10) Pub. No.: US 2024/0398830 A1
(43) Pub. Date: Dec. 5, 2024

(54) COMPOSITIONS AND USES THEREOF FOR TREATMENT OF NEUROLOGICAL DISORDERS

(71) Applicant: UNIVERSITY OF SOUTH FLORIDA, Tampa, FL (US)

(72) Inventors: Charles Joseph LOCKWOOD, Tampa, FL (US); Laura BLAIR, Tampa, FL (US); Ozlem GUZELOGLU-KAYISLI, Wesley Chapel, FL (US); Umit Ali KAYISLI, Wesley Chapel, FL (US)

(21) Appl. No.: 18/698,748

(22) PCT Filed: Oct. 5, 2022
(86) PCT No.: PCT/US2022/077578

§ 371 Date: Apr. 4, 2024

Related U.S. Application Data
(60) Provisional application No. 63/252,343, filed on Oct. 5, 2021.

(51) Int. Cl. A61K 31/5575; A61K 31/201

(52) U.S. Cl. CPC A61K 31/5575; A61K 31/201

(57) ABSTRACT

Disclosed are compositions and methods for delaying or preventing neurological disorders.

### CROSS-REFERENCE TO RELATED APPLICATIONS

This application claims the benefit of U.S. Provisional Application No. 63/252,343, filed Oct. 5, 2021, which is incorporated by reference herein in its entirety.

### FIELD

The present disclosure relates to the field of treatment of neurological disorders.

### BACKGROUND

In 2011, nearly 100 million Americans were affected by at least one neurological disorder. This translates into an overall cost of $765 billion for the more prevalent conditions. Alzheimer's disease, a neurodegenerative disorder, is the sixth leading cause of death in the United States. It is estimated that 5.5 million Americans age 65 and older have dementia caused by Alzheimer's disease, while treatment remains a challenge as the current approved drugs by FDA treat only symptoms and not the underlying destruction of nerve cells. What are needed are new compositions and methods for treating neurological disorders. The compositions and methods disclosed herein address these and other needs.

### SUMMARY

In accordance with the purposes of the disclosed materials and methods, as embodied and broadly described herein, the disclosed subject matter, in one aspect, relates to compounds, compositions and methods of making and using compounds and compositions. In specific aspects, the disclosed subject matter relates to methods for treating a neurological disorder.

The details of one or more embodiments of the invention are set forth in the accompanying drawings and the description below. Other features, objects, and advantages of the invention will be apparent from the description and drawings, and from the claims.

Disclosed herein is a method of treating or preventing a neurological disorder in a subject, comprising administering to the subject a therapeutically effective amount of a composition comprising 15-deoxy-Δ12,14-prostaglandin J2, 9-nitro-9E-octadecenoic acid, 10-nitro-9E-octadecenoic acid, a derivative thereof, or a combination thereof. In one example, the composition comprises 9-nitro-9E-octadecenoic acid. In one example, the composition comprises 10-nitro-9E-octadecenoic acid. In one example, the composition comprises 15-deoxy-Δ12,14-prostaglandin J2.

In some embodiments, the composition is administered through an oral route, intravenously, or intracranially.

In some embodiments, the neurological disorder is selected from the group consisting of depression, post-traumatic stress disorder (PTSD), anxiety, and Alzheimer's disease. In some embodiments, the subject has an increased level of FK506-binding protein (FKBP51) relative to a reference control.

In some embodiments, the composition decreases a level of FKBP51 in a biological sample derived from the subject in comparison to a control. In some embodiments, the biological sample is a neural tissue.

Also disclosed herein is a method treating a neurological disorder in a subject, comprising
a) determining whether a biological sample obtained from the subject has an increased level of FKBP51 as compared to a control; and
b) if the subject has an increased level of FKBP51 as compared to the control administering to the subject a therapeutically effective amount of a composition comprising 15-deoxy-Δ12,14-prostaglandin J2, 9-nitro-9E-octadecenoic acid, 10-nitro-9E-octadecenoic acid, a derivative thereof, or a combination thereof.

In some embodiments, the biological sample is a neural tissue.

### BRIEF DESCRIPTION OF DRAWINGS

The accompanying figures, which are incorporated in and constitute a part of this specification, illustrate several aspects described below.

FIGS. 1A-1B show qPCR (FIG. 1A) and immunoblot analysis (FIG. 1B) of FKBP51 levels in cultures of H4 human neuroglioma cell line treated with methyl acetate vehicle (MeOAc) or ethanol vehicle (ETOH) or 10 μM 15-deoxy-12,14-prostaglandin J2 (J2) or 10 μM 9-nitro oleic acid (9N), or 10 μM 10-nitro oleic acid (ION) in control (dimethyl sulfoxide; DMSO) or dexamethasone (Dex) containing media for 6 h and 24 h. Cultures incubated with treatments for 6 h were used for RNA analysis whereas cultures incubated with treatments for 24 h were used for protein analysis. Bars represent mean±SEM; n=3 independent experiments.

FIG. 2 shows immunoblot analysis of FKBP51 levels in HT-22 mouse hippocampal neuronal cell line cultures treated with ethanol vehicle (EtOH Veh) or methyl acetate vehicle (Me-Acetate Veh) or 20 μM 15-deoxy-12,14-prostaglandin J2 (J2) or 20 μM 9-nitro oleic acid (9N), or 20 μM 10-nitro oleic acid (ION) in control (dimethyl sulfoxide; DMSO) or 500 nM dexamethasone (Dex) containing media.

FIG. 3 shows luciferase assay of glucocorticoid receptor response element (GRE) in vehicle (Veh) or 15-deoxy-12,14-prostaglandin J2 (J2)+nitro oleic acid (9N) or +10-nitro oleic acid (ION) in cultures of HeLa cells transfected with empty vector (EV) or FKBP51 vector (51).

FIG. 4 shows chemical structures of 15-deoxy-12,14-prostaglandin J2, 9-nitro oleic acid and 10-nitro oleic acid.

### DETAILED DESCRIPTION

Terms used throughout this application are to be construed with ordinary and typical meaning to those of ordinary skill in the art. However, Applicant desires that the following terms be given the particular definition as defined below.

### Terminology

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of ±20%, ±10%, ±5%, or ±1% from the measurable value.

“Activate”, “activating”, and “activation” mean to increase an activity, response, condition, or other biological parameter. This may also include, for example, a 10% increase in the activity, response, or condition, as compared to the native or control level. Thus, the increase can be a 1, 5, 10, 20, 30, 40, 50, 60, 70, 80, 90, 100%, or any amount of increase in between as compared to native or control levels.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

The term “biological sample” as used herein means a sample of biological tissue or fluid. Such samples include, but are not limited to, tissue isolated from animals. Biological samples can also include sections of tissues such as biopsy and autopsy samples, frozen sections taken for histologic purposes, blood, plasma, serum, sputum, stool, tears, mucus, hair, and skin. Biological samples also include explants and primary and/or transformed cell cultures derived from patient tissues. A biological sample can be provided by removing a sample of cells from an animal, but can also be accomplished by using previously isolated cells (e.g., isolated by another person, at another time, and/or for another purpose), or by performing the methods as disclosed herein in vivo. Archival tissues, such as those having treatment or outcome history can also be used.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like.

A “control” is an alternative subject or sample used in an experiment for comparison purposes. A control can be “positive” or “negative.”

“Decrease” can refer to any change that results in a lower level of gene expression, protein expression, amount of a symptom, disease, composition, condition, or activity. A substance is also understood to decrease the level of the gene, the protein, the composition, or the amount of the condition when the level of the gene, the protein, the composition, or the amount of the condition is less/lower relative to the output of the level of the gene, the protein, the composition, or the amount of the condition without the substance. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

“Increase” can refer to any change that results in a higher level of gene expression, protein expression, amount of a symptom, disease, composition, condition, or activity. A substance is also understood to increase the level of the gene, the protein, the composition, or the amount of the condition when the level of the gene, the protein, the composition, or the amount of the condition is more/higher relative to the output of the level of the gene, the protein, the composition, or the amount of the condition without the substance. Also, for example, an increase can be a change in the symptoms of a disorder such that the symptoms are less than previously observed. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 1, 5, 10, 20, 30, 40, 50, 60, 70, 80, 90, 100%, or any amount of reduction in between as compared to native or control levels.

“Inhibitors” and “activators” of expression or of activity are used to refer to inhibitory or activating molecules, respectively, identified using in vitro and in vivo assays for expression or activity of a described target protein, e.g., ligands, agonists, antagonists, and their homologs and mimetics. Inhibitors are agents that, e.g., inhibit expression or bind to, partially or totally block stimulation or protease activity, decrease, prevent, delay activation, inactivate, desensitize, or down regulate the activity of the described target protein. Activators are agents that, e.g., induce or activate the expression of a described target protein or bind to, stimulate, increase, open, activate, facilitate, enhance activation or protease inhibitor activity, sensitize or up regulate the activity of described target protein (or encoding polynucleotide). Inhibition of a described target protein is achieved when the activity value relative to the control is about 80%, optionally 50% or 25, 10%, 5% or 1%. Activation of the described target protein is achieved when the activity value relative to the control is 110%, optionally 150%, optionally 200, 300%, 400%, 500%, or 1000-3000% or more.

As used herein, the term “neurological disorder” refers to a disease or disorder in the central nervous system (CNS, brain, and spinal cord), and includes, but is not limited to, depression, post-traumatic stress disorder (PTSD), anxiety, and Alzheimer's disease.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

The term “subject” refers to a human in need of treatment for any purpose, and more preferably a human in need of treatment. The term “subject” can also refer to non-human animals, such as non-human primates.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

“Therapeutically effective amount” or “therapeutically effective dose” of a composition (e.g., a composition comprising an agent) refers to an amount that is effective to achieve a desired therapeutic result. In some embodiments, a desired therapeutic result is the delay and/or prevention of a neurological disorder (e.g., depression, post-traumatic stress disorder (PTSD), anxiety, and Alzheimer's disease). In some embodiments, a desired therapeutic result is the treatment and/or prevention of a neurological disorder (e.g., depression, post-traumatic stress disorder (PTSD), anxiety, and Alzheimer's disease). In some embodiment, a desired therapeutic result is a reduction of FK506-binding protein (FKBP51) levels in the subject with a neurological disorder (e.g., depression, post-traumatic stress disorder (PTSD), anxiety, and Alzheimer's disease). In some embodiment, a desired therapeutic result is a reduced level of a beta-amyloid protein or a reduced accumulation of a beta-amyloid protein in the subject. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

### Compounds and Methods of Treatment

Single nucleotide polymorphisms in FKBP5 gene, coding for FKBP51, can combine with stress to elevate FKBP51 levels and increase risk for major depression, post-traumatic stress disorder (PTSD), and anxiety disorders. FKBP51 levels also increase with age and are further elevated in the brains of Alzheimer's disease (AD) patients. Disclosed herein are 15-deoxy-Δ12,14-prostaglandin J2, 9-nitro-9E-octadecenoic acid, 10-nitro-9E-octadecenoic acid or a derivative thereof and their effects on the prevention of the up-regulation of neuronal FKBP51 levels and decreasing the neuronal FKBP51 levels, such as following stress. Thus, ablating FKBP51 by these fatty acid derivatives or their synthetically modified forms in patient groups with a neurological disorder can benefit from the composition disclosed herein.

Accordingly, disclosed herein is method of treating or preventing a neurological disorder in a subject, comprising administering to the subject a therapeutically effective amount of a composition comprising 15-deoxy-Δ12,14-prostaglandin J2, 9-nitro-9E-octadecenoic acid, 10-nitro-9E-octadecenoic acid, or a derivative thereof, or a combination thereof. In some embodiments, the subject (e.g., a biological sample obtained from the subject) has an increased level of FK506-binding protein (FKBP51) relative to a reference control.

Also, disclosed herein is a method of treating or preventing a neurological disorder in a subject, comprising
a) determining whether a biological sample obtained from the subject has an increased level of FKBP51 as compared to a reference control; and
b) administering to the subject a therapeutically effective amount of a composition comprising 15-deoxy-Δ12,14-prostaglandin J2, 9-nitro-9E-octadecenoic acid, 10-nitro-9E-octadecenoic acid, or a derivative thereof, or a combination thereof if the biological sample has an increased level of FKBP51 as compared to the reference control.

In some embodiments, the method further comprises a step of obtaining a biological sample from the subject prior to step a).

In some embodiments, the reference control is a healthy control or a study population.

In some embodiments, the level of FKBP51 (a level of FKBP51 mRNA and/or a level of FKBP1 protein) in the subject (e.g., a biological sample obtained from the subject) is at least about 5% (e.g., at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1000%, at least about 2000%, at least about 3000%, or at least about 5000%) or at least about 5 times (e.g., at least about 6 times, at least about 7 times, at least about 8 times, at least about 9 times, at least about 10 times, at least about 20 times, at least about 30 times, at least about 40 times, at least about 50 times, or at least about 100 times) higher as compared to a subject in general or a study population. In some embodiments, the biological sample is a neuronal tissue, a nerve biopsy sample, a cerebrospinal fluid sample, or a blood sample.

“FK506 binding protein 51” or “FKBP51” is a protein that in humans is encoded by the FKBP5 gene. In some embodiments, the FKBP51 polypeptide is that identified in one or more publicly available databases as follows: HGNC: 3721, Entrez Gene: 2289, Ensembl: ENSG00000096060, OMIM: 602623, UniProtKB: Q13451. In some embodiments, the FKBP51 polypeptide comprises the sequence of SEQ ID NO: 1, or a polypeptide sequence having at or greater than about 80%, about 85%, about 90%, about 95%, or about 98% identity with SEQ ID NO: 1, or a polypeptide comprising a portion of SEQ ID NO: 1 that is a functional fragment of FKBP51. The FKBP51 polypeptide of SEQ ID NO: 1 may represent an immature or pre-processed form of mature FKBP51, and accordingly, included herein are mature or processed portions of the FKBP51 polypeptide in SEQ ID NO: 1. In some embodiments, the FKBP51 mRNA comprises an mRNA sequence encoding the FKBP51 protein disclosed herein.

In some embodiments, the composition disclosed herein comprises 9-nitro-9E-octadecenoic acid. In some embodiments, the composition comprises 10-nitro-9E-octadecenoic acid. In some embodiments, the composition comprises 15-deoxy-Δ12,14-prostaglandin J2. In some embodiments, the composition disclosed herein comprises 9-nitro-9E-octadecenoic acid, 10-nitro-9E-octadecenoic acid, 15-deoxy-Δ12,14-prostaglandin J2, or a combination thereof. In some embodiments, the composition comprises 15-deoxy-Δ12,14-prostaglandin J2, 9-nitro-9E-octadecenoic acid, and 10-nitro-9E-octadecenoic acid.

In some embodiments, the method disclosed herein comprises administering to the subject a therapeutically effective amount of 15-deoxy-Δ12,14-prostaglandin J2 and 9-nitro-9E-octadecenoic acid. In some embodiments, the method disclosed herein comprises administering to the subject a therapeutically effective amount of 15-deoxy-Δ12,14-prostaglandin J2 and 10-nitro-9E-octadecenoic acid. In some embodiments, the method disclosed herein comprises administering to the subject a therapeutically effective amount of 9-nitro-9E-octadecenoic acid and 10-nitro-9E-octadecenoic acid. In some embodiments, the method disclosed herein comprises administering to the subject a therapeutically effective amount of 15-deoxy-Δ12,14-prostaglandin J2, 9-nitro-9E-octadecenoic acid, and 10-nitro-9E-octadecenoic acid.

In some embodiments, the composition disclosed herein is formulated in a pharmaceutically acceptable carrier.

In some embodiments, wherein the composition decreases a level of FKBP51 in a biological sample derived from the subject in comparison to a control, wherein the control can be a subject not administered the composition or as compared with the FKBP51 level in a study population. In some embodiments, the decrease is at least about 5% (e.g., at least 10%, at least about 20%, at least about 30%, at least about 40%, at least about 50%, at least about 60%, at least about 70%, at least about 80%, at least about 90%, at least about 95%) as compared to the control. In some embodiments, the composition decreases a mRNA level or a protein level of FKBP1 in the biological sample.

In some embodiments, the biological sample is a neuronal tissue, a nerve biopsy sample, a cerebrospinal fluid sample, or a blood sample.

“Alzheimer's disease” or “AD” as used herein refers to all form of dementia, identified as a degenerative and terminal cognitive disorder. The disease may be static, the result of a unique global brain injury, or progressive, resulting in long-term decline in cognitive function due to damage or disease in the body beyond what might be expected from normal aging. Particularly, it has been identified that Alzheimer's disease is caused by the accumulation of the beta-amyloid protein, or AD, and it has been known that the induction of the degeneration and death of nerve cells caused by the amyloid protein is involved in the mechanism of Alzheimer's disease. The beta-amyloid protein involved in Alzheimer's has several different molecular forms that collect between neurons. One form, beta-amyloid 42, is thought to be especially toxic. As noted above, FKBP51 levels increase with age and are further elevated in the brains of Alzheimer's disease (AD) patients.

It should be understood that a treatment of Alzheimer's disease may be a treatment of one or more of memory loss, poor judgment leading to bad decisions, loss of spontaneity and sense of initiative, repeating questions, having difficulties to organize thoughts, mood and personality changes, and/or increased anxiety and/or aggression. Treatment can be indicated by one or more of mental status and neuropsychological testing indicating improvement in memory, mitigation of memory loss, and/or improvement in other thinking skills, and/or brain imaging (e.g., using magnetic resonance imaging (MRI), computerized tomography (CT), or positron emission tomography (PET)) indicating mitigation of brain shrinkage, amyloid deposits, or neurofibrillary tangles, improvement in nutrient metabolism in brain, inhibition of an increase of a FKBP51 level in a biological sample, and/or decreasing a FKBP51 level in a biological sample as compared with prior to treatment of the subject or as compared with the incidence of such symptom in a study population.

“Depression” or “major depressive disorder” refers to a mood disorder that causes a persistent feeling of sadness and loss of interest. It should be understood that a treatment of depression may be a treatment of one or more of change in depressed mood or loss of interest and pleasure, indifference or apathy, or change in a number of neurovegetative functions (for example, sleep patterns, appetite and body weight, motor agitation or retardation, or fatigue), impairment in concentration and decision making, constant feelings of shame or guilt, and thoughts of death or dying. Treatment can be indicated by one or more of psychiatric evaluation indicating improved feelings and thoughts, and/or criteria listed on Diagnostic and Statistical Manual of Mental Disorders (DSM-5), published by the American Psychiatric Association, indicating mitigation of depression as compared with prior to treatment of the subject or as compared with the incidence of such symptom in a study population. In some examples, treatment can be indicated by inhibition of an increase of a FKBP51 level in a biological sample, and/or decreasing a FKBP51 level in a biological sample as compared with prior to treatment of the subject or as compared with the incidence of such symptom in a study population.

“Post-traumatic stress disorder” or “PTSD” refers to a psychiatric disorder that can be triggered by a terrifying event, either experiencing it or witnessing it. Treatment of PTSD can be indicated by mitigation of flashbacks, nightmares, anxiety, negative changes in thinking and mood, an inhibition of an increase of a FKBP51 level in a biological sample, and/or decreasing a FKBP51 level in a biological sample as compared with as compared with prior to treatment of the subject or as compared with the incidence of such symptom in a study population.

In some embodiments, the composition described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavemous, intrathecal, intravitreal, intracerebral, gingival, subgingival, intracerebroventricular, and intradermal. Such formulations may be prepared by any method known in the art.

The disclosed methods can be performed any time prior to the onset of a neurological disorder. In one aspect, the disclosed methods can be employed 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 years; 12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours prior to the onset of a neurological disorder; or 1, 2, 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 45, or 60 years after the onset of a neurological disorder.

Dosing frequency for the composition of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, daily, two times per day, three times per day, four times per day, five times per day, six times per day, eight times per day, nine times per day, ten times per day, eleven times per day, twelve times per day, once every 12 hours, once every 10 hours, once every 8 hours, once every 6 hours, once every 5 hours, once every 4 hours, once every 3 hours, once every 2 hours, once every hour, once every 40 min, once every 30 min, once every 20 min, or once every 10 min. Administration can also be continuous and adjusted to maintaining a level of the compound within any desired and specified range.

Dosages are typically modified according to the characteristics of the subject (weight, gender, age, etc.), severity of disease (e.g., degree of reduced ciliation), specifics and purity of the active agent to be administered, route of administration, nature of the formulation, and numerous other factors. Generally, the active agent (e.g., 15-deoxy-Δ12,14-prostaglandin J2, 9-nitro-9E-octadecenoic acid, 10-nitro-9E-octadecenoic acid, or a derivative thereof) is administered to the subject at a dosage ranging from 0.1 μg/kg body weight to 100 g/kg body weight. In some embodiments, the active agent is administered to the subject at a dosage of from 1 μg/kg to 10 g/kg, from 10 μg/kg to 1 g/kg, from 10 μg/kg to 500 mg/kg, from 10 μg/kg to 100 mg/kg, from 10 μg/kg to 10 mg/kg, from 10 μg/kg to 1 mg/kg, from 10 μg/kg to 500 μg/kg, or from 10 μg/kg to 100 μg/kg body weight. The dosage of administration for the active agent disclosed herein can be from about 0.01 mg/kg body weight to about 100 mg/kg body weight. In some examples, the dosage is about 0.01 mg/kg body weight, about 0.05 mg/kg body weight, about 0.1 mg/kg body weight, about 0.5 mg/kg body weight, about 1 mg/kg body weight, about 1.5 mg/kg body weight, about 2 mg/kg body weight, about 2.5 mg/kg body weight, about 3 mg/kg body weight, about 3.5 mg/kg body weight, about 4 mg/kg body weight, about 4.5 mg/kg body weight, about 5 mg/kg body weight, about 5.5 mg/kg body weight, about 6 mg/kg body weight, about 6.5 mg/kg body weight, about 7 mg/kg body weight, about 7.5 mg/kg body weight, about 8 mg/kg body weight, about 8.5 mg/kg body weight, about 9 mg/kg body weight, about 9.5 mg/kg body weight, about 10 mg/kg body weight, about 11 mg/kg body weight, about 12 mg/kg body weight, about 13 mg/kg body weight, about 14 mg/kg body weight, about 15 mg/kg body weight, about 20 mg/kg body weight, about 25 mg/kg body weight, about 30 mg/kg body weight, about 35 mg/kg body weight, about 40 mg/kg body weight, about 45 mg/kg body weight, about 50 mg/kg body weight, about 55 mg/kg body weight, about 60 mg/kg body weight, about 65 mg/kg body weight, about 70 mg/kg body weight, about 75 mg/kg body weight, about 80 mg/kg body weight, about 85 mg/kg body weight, about 90 mg/kg body weight, about 95 mg/kg body weight, or about 100 mg/kg body weight. Dosages above or below the range cited above may be administered to the individual patient if desired.

### EXAMPLES

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. While the invention has been described with reference to particular embodiments and implementations, it will be understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. Such equivalents are intended to be encompassed by the following claims. It is intended that the invention not be limited to the particular implementations disclosed herein, but that the invention will include all implementations falling within the scope of the appended claims.

### Example 1. FKBP51 and Neurological Disorders

The 51 kDa FK506-binding protein (FKBP51) is an important co-chaperone of the 90 kDa heat shock protein (Hsp90) machinery. Common single nucleotide polymorphisms (SNPs) in the gene coding for FKBP51, FKBP5, can combine with stress to elevate levels of FKBP51 through glucocorticoid response elements (GREs) and increase susceptibility for major depression, post-traumatic stress disorder (PTSD), and anxiety disorders. Furthermore, FKBP51 levels increase with age and are further elevated in the brains of Alzheimer's disease (AD) patients. Importantly, mice lacking FKBP51 are viable and show protection from stress-related phenotypes.

These findings have called attention to FKBP51 as a target for the treatment of these disorders. There are no current treatments available in the clinic that target this protein. Three fatty acid (lipid/prostaglandin) derivatives were recently identified for preventing the upregulation of FKBP51 following stress. These lipid-compounds are naturally found in the body, which can accelerate their ability to be implemented into clinics to treat specific patient populations with high levels of FKBP51. Patients with elevated levels of FKBP51, specifically those with SNPs in the FKBP5 gene or major depression and/or anxiety disorders or PTSD or aged/AD patients, can benefit from this discovery by ablating stress induced FKBP51 by use of either these fatty acid derivatives or their synthetically modified forms.

### Example 2. The Three Fatty Acids Inhibit Dexamethasone Induced FKBP51 Protein Levels in Neural Cells

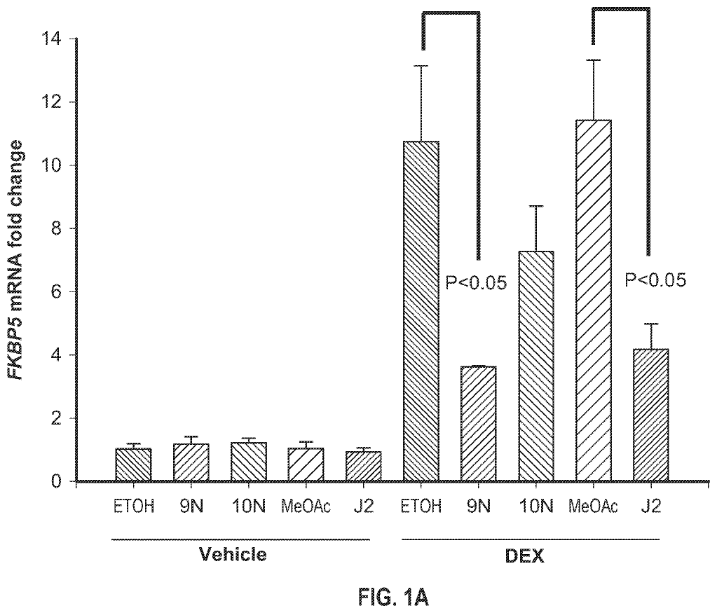

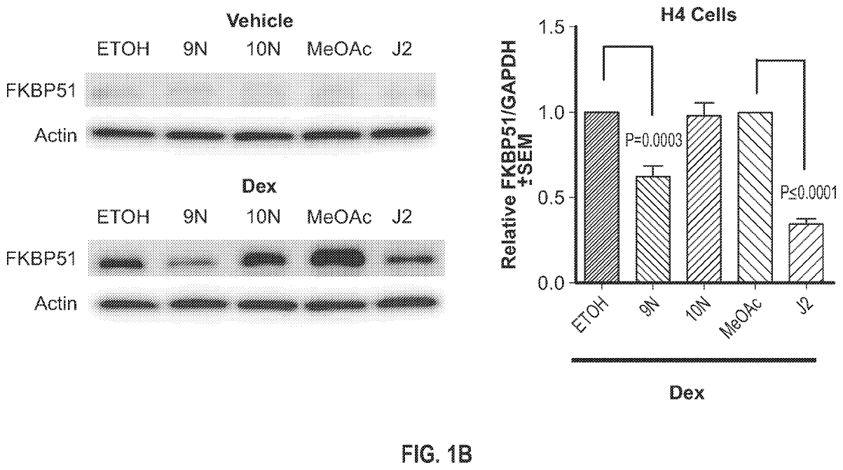

In Cultures of H4 Human Neuroglioma Cell Line, 15-Deoxy-12,14-Prostaglandin J2, 9-Nitro oleic acid and 10-nitro oleic acid treatments inhibit dexamethasone induced FKBP51 protein levels (FIG. 1).

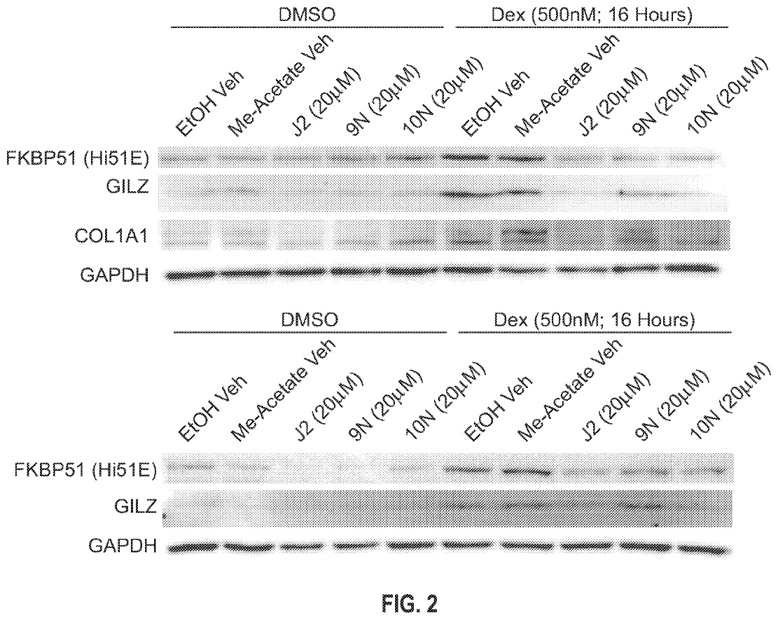

In cultures of HT-22 mouse hippocampal neuronal cell line, 9-nitro oleic acid, 10-nitro oleic acid and 15-deoxy-12,14-prostaglandin J2 treatments inhibit dexamethasone induced FKBP51 protein levels (FIG. 2).

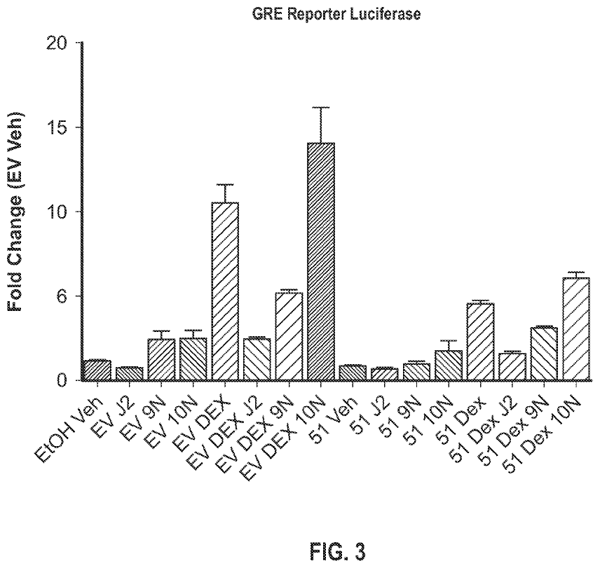

GRE reporter luciferase assay identified that 9-nitro oleic acid, 10-nitro oleic acid and 15-deoxy-12,14-prostaglandin treatments alter glucocorticoid receptor response in dexamethasone treated HeLa cells (FIG. 3).

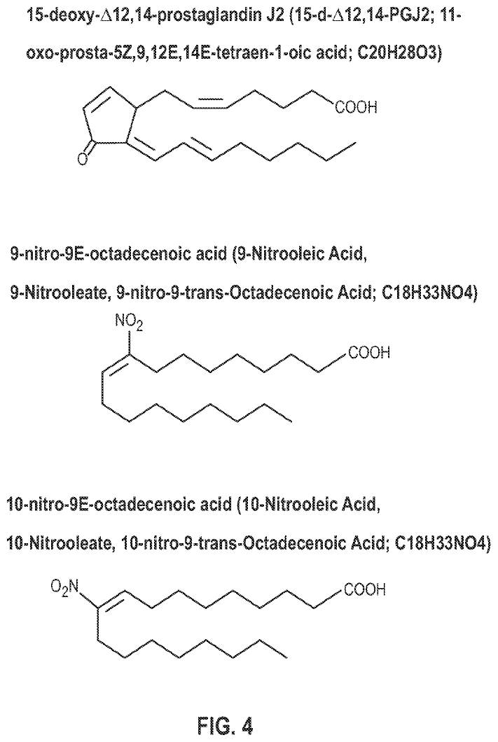

The synthetically modified versions of these compounds are developed. It was found that besides their common effects to inhibit FKBP51 expression, they have similar core structures. Therefore, the design of new chemical modifications of any of the three compounds have therapeutic actions in patients with depression, PTSD, anxiety disorders and/or AD patients. The nitro group is a common feature of 9-nitro oleic acid and 10-nitro oleic acid, but not in 15-deoxy-12,14-prostaglandin J2. Moreover, the C═C double bonds at the 5-, 12- and 14-positions and 11-oxo group are present in 15-deoxy-12,14-prostaglandin J2, but not in 9-nitro oleic acid and 10-nitro oleic acid. Therefore, further synthetic modifications of these compounds including, but not limited to the addition, removal, or rearrangements of the oxo groups and/or nitro groups in their chemical formula can enhance their therapeutic potentials in these patient populations. There is greater therapeutic potential to treat depression, PTSD, anxiety disorders and/or AD patients by numerous other synthetic modifications such as acetylation, hydroxylation within or with an expanding chemical framework of 9-nitro oleic acid and 10-nitro oleic acid or 15-deoxy-12,14-prostaglandin J2 (FIG. 4).

SEQUENCES

SEQ ID NO: 1
MTTDEGAKNNEESPTATVAEQGEDITSKKDRGVLKIVKRVGNGEETPMI
GDKVYVHYKGKLSNGKKFDSSHDRNEPFVFSLGKGQVIKAWDIGVATMK
KGEICHLLCKPEYAYGSAGSLPKIPSNATLFFEIELLDFKGEDLFEDGG
IIRRTKRKGEGYSNPNEGATVEIHLEGRCGGRMFDCRDVAFTVGEGEDH
DIPIGIDKALEKMQREEQCILYLGPRYGFGEAGKPKFGIEPNAELIYEV
TLKSFEKAKESWEMDTKEKLEQAAIVKEKGTVYFKGGKYMQAVIQYGKI
VSWLEMEYGLSEKESKASESFLLAAFLNLAMCYLKLREYTKAVECCDKA
LGLDSANEKGLYRRGEAQLLMNEFESAKGDFEKVLEVNPQNKAARLQIS
MCQKKAKEHNERDRRIYANMFKKFAEQDAKEEANKAMGKKTSEGVTNEK
GTDSQAMEEEKPEGHV

1. A method of treating a neurological disorder in a subject, comprising administering to the subject a therapeutically effective amount of a composition comprising 15-deoxy-Δ12,14-prostaglandin J2, 9-nitro-9E-octadecenoic acid, 10-nitro-9E-octadecenoic acid, a derivative thereof, or a combination thereof.

2. The method of claim 1, wherein the composition comprises 9-nitro-9E-octadecenoic acid.

3. The method of claim 1, wherein the composition comprises 10-nitro-9E-octadecenoic acid.

4. The method of claim 1, wherein the composition comprises 15-deoxy-Δ12,14-prostaglandin J2.

5. The method of claim 1, wherein the neurological disorder is selected from the group consisting of depression, post-traumatic stress disorder (PTSD), anxiety, and Alzheimer's disease.

6. The method of claim 1, wherein the subject has an increased level of FK506-binding protein (FKBP51) relative to a reference control.

7. The method of claim 1, wherein the composition decreases a level of FKBP51 in a biological sample derived from the subject in comparison to a control.

8. The method of claim 7, wherein the biological sample is a neuronal tissue, a nerve biopsy sample, a cerebrospinal fluid sample, or a blood sample.

9. The method of claim 1, wherein the composition is administered through an oral route, intracranially, or intravenously.

10. The method of claim 1, wherein the subject is a human.

11. A method of treating a neurological disorder in a subject, comprising
a) determining whether a biological sample obtained from the subject has an increased level of FKBP51 as compared to a control; and
b) administering to the subject a therapeutically effective amount of a composition comprising 15-deoxy-Δ12,14-prostaglandin J2, 9-nitro-9E-octadecenoic acid, 10-nitro-9E-octadecenoic acid, a derivative thereof, or a combination thereof if the subject has an increased level of FKBP51 as compared to the control.

12. The method of claim 11, further comprising a step of obtaining a biological sample from the subject prior to step a).

13. The method of claim 11, wherein the composition comprises 9-nitro-9E-octadecenoic acid.

14. The method of claim 11, wherein the composition comprises 10-nitro-9E-octadecenoic acid.

15. The method of claim 11, wherein the composition comprises 15-deoxy-Δ12,14-prostaglandin J2.

16. The method of claim 11, wherein the neurological disorder is selected from the group consisting of depression, post-traumatic stress disorder (PTSD), anxiety, and Alzheimer's disease.

17. The method of claim 11, wherein the biological sample is a neuronal tissue, a nerve biopsy sample, a cerebrospinal fluid sample, or a blood sample.

18. The method of claim 11, wherein the composition is administered through an oral route, intracranially, or intravenously.

19. The method of claim 11, wherein the subject is a human.
